(19) United States
(12) Patent Application Publication
(10) Pub. No.: US 2026/0022973 A1
(43) Pub. Date: Jan. 22, 2026

(54) SYSTEMS FOR REAL-TIME LASER POWER MONITORING

(71) Applicant: Alcon Inc., Fribourg (CH)

(72) Inventors: David Jung, Tustin, CA (US); Keith Watanabe, Irvine, CA (US); Mikhail Ovchinnikov, Dana Point, CA (US)

(21) Appl. No.: 19/262,500

(22) Filed: Jul. 8, 2025

Related U.S. Application Data
(60) Provisional application No. 63/673,820, filed on Jul. 22, 2024.

(51) Int. Cl. G01J 11/00; A61F 9/008; G01D 1/02; G01J 1/18

(52) U.S. Cl. CPC G01J 11/00; G01D 1/02; G01J 1/18; A61F 9/008; A61F 2009/00844

(57) ABSTRACT

A system including a first laser source configured to generate a first laser beam in a pulsed form, and a second laser source configured to generate a second laser beam. The system further includes an optical fiber configured to: receive the first laser beam, output the first laser beam from a distal end of the optical fiber; receive the second laser beam, output the second laser beam from a distal end of the optical fiber, receive a reflected portion of the second laser beam that is reflected back into the distal end of the optical fiber, and direct the reflected portion of the second laser beam to the optical detector. The optical detector is configured to receive the reflected portion of the second laser beam from the optical fiber and generate an optical detector output based on the reflected portion of the second laser beam.

### INTRODUCTION

To prepare a surgical laser system for surgery, components of the surgical laser system (e.g., a surgical console with a laser source, handpiece, consumable, optical fiber, etc.) are all assembled together. Proper assembly allows for efficient transmission of a laser light from the laser source all the way to the optical fiber tip. An incomplete and/or incorrect assembly or misalignment of components during use can lead to a lack of sufficient radiation at the optical fiber tip and, thus, a lack of efficiency. Moreover, in cases where there is an incomplete and/or incorrect assembly or misalignment of components, while the power at the source of the laser may be known, the power level at the optical fiber tip may be unknown due to inherent variability in the optical connection of the different components of the surgical laser system during assembly or through use.

Conventional systems require the use of a power meter coupled to the surgical laser system to measure power levels of transmitted laser light. Typically, the power level of the laser light transmitted by the surgical laser system is measured prior to a surgical procedure. However, because the power meter is a non-sterile piece of equipment, the use of the power meter in an operating room environment is cumbersome and time consuming as the non-sterile power meter must be carefully handled and isolated to maintain sterility for patient safety. Furthermore, because conventional testing systems are non-sterile, use to verify laser power delivery during surgery is not possible.

### SUMMARY

The present disclosure generally relates to systems and methods for laser power measurement, and more particularly, to systems and methods for laser power measurement in operating environments (e.g., sterile surgical operating environments).

In certain embodiments, a system includes a first laser source configured to generate a first laser beam in a pulsed form, and a second laser source configured to generate a second laser beam. The system further includes an optical fiber configured to: receive the first laser beam from the first laser source; output the first laser beam from a distal end of the optical fiber; receive the second laser beam from the second laser source; output the first laser beam from a distal end of the optical fiber; receive a reflected portion of the second laser beam that is reflected back into the distal end of the optical fiber; and direct the reflected portion of the second laser beam to the optical detector. The optical detector is configured to receive the reflected portion of the second laser beam from the optical fiber and generate an optical detector output based on the reflected portion of the second laser beam. The system further includes a controller configured to determine one or more widths of one or more pulses in the optical detector output and determine an estimated energy for one or more pulses in the first laser beam based on the one or more widths.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope and may admit to other equally effective embodiments.

FIG. 1A illustrates an example arrangement of a system for performing laser power measurement, in accordance with certain embodiments of the present disclosure.

FIG. 1B illustrates another example arrangement of a system for performing laser power measurement, in accordance with certain embodiments of the present disclosure.

FIG. 2 illustrates an example method of performing laser power measurement, in accordance with certain embodiments of the present disclosure.

FIGS. 3A-3F illustrate a bubble progression during performance of laser power measurement, in accordance with certain embodiments of the present disclosure.

FIGS. 4 and 5 illustrate graphs of reflected portions of laser light, in accordance with certain embodiments of the present disclosure.

FIG. 6 illustrates a system for processing sampled back-reflected light to derive delivered pulse energy, in accordance with certain embodiments of the present disclosure.

FIG. 7 is a timing diagram illustrating the generation of laser pulses, in accordance with certain embodiments of the present disclosure.

FIG. 8 is a timing diagram illustrating the acquisition of samples of measurements of back reflected light, in accordance with certain embodiments of the present disclosure.

FIG. 9 is a plot of measured back-reflected light prior to processing, in accordance with certain embodiments of the present disclosure.

FIG. 10 is a plot of measured back-reflected light following processing, in accordance with certain embodiments of the present disclosure.

FIG. 11 is a plot comparing estimated pulse energy based on pulse width compared to actual pulse energy, in accordance with certain embodiments of the present disclosure.

FIG. 12 illustrates a schematic diagram of a surgical console and components thereof, in accordance with certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure generally relates to systems and methods for laser power measurement, and more particularly, to systems and methods for laser power measurement in operating environments (e.g., sterile surgical operating environments).

As described above, conventional systems require the use of a power meter to measure the power of laser beams delivered from the working tip of an optical fiber. However, because power meters are typically non-sterile pieces of equipment, the use of a power meter in an operating room environment is cumbersome and time consuming when trying to maintain sterility for patient safety. Further, due to the non-sterile nature of power meters, the power levels of laser beams transmitted by a surgical laser system are typically only measured prior to performance of a surgical procedure. Yet, it is important to monitor the power of laser beams delivered from the working tip of an optical fiber throughout the performance of a surgical procedure. Accordingly, embodiments of the present disclosure provide systems and methods that enable efficient laser power measurement before, during, and after performance of a surgical procedure in a sterile operating environment.

In certain embodiments described herein, power measurement of a surgical laser system is performed utilizing two laser sources: 1) a first laser source configured to generate a first laser beam (a “treatment” laser beam, such as an infrared (IR) laser beam); and 2) a second laser source configured to generate a second laser beam (a “test” laser beam, such as a visible light laser beam). Prior to or during a surgical procedure, the first laser beam and second laser beam may be propagated into a test material (e.g., water, saline, balanced salt solution, gel, etc.) to measure or monitor power levels of the first laser beam generated by the first laser source. The first laser beam, when propagated into the test material, generates a transient vapor bubble in the test material, which may alter the portion of the second laser beam reflected back into the surgical laser system. The duration of the change in the reflected portion of the second laser beam may then be optically measured to determine a lifetime of the vapor bubble, which can be correlated with laser power values based on one or more correlation curves.

Laser power measurement using the back-reflection of the second laser beam allows for efficient testing in a sterile environment and even during a surgical procedure, without necessitating the utilization of non-sterile equipment such as a power meter. Testing of laser power levels facilitates identification of equipment issues including improper assembly, faulty components, misalignment due to movement or use prior to or during a surgical operation, and the like. Thus, the methods and systems described herein enable not only real-time testing during surgical procedures, but also improve overall safety of the surgical procedures by decreasing the risk of introducing contaminants.

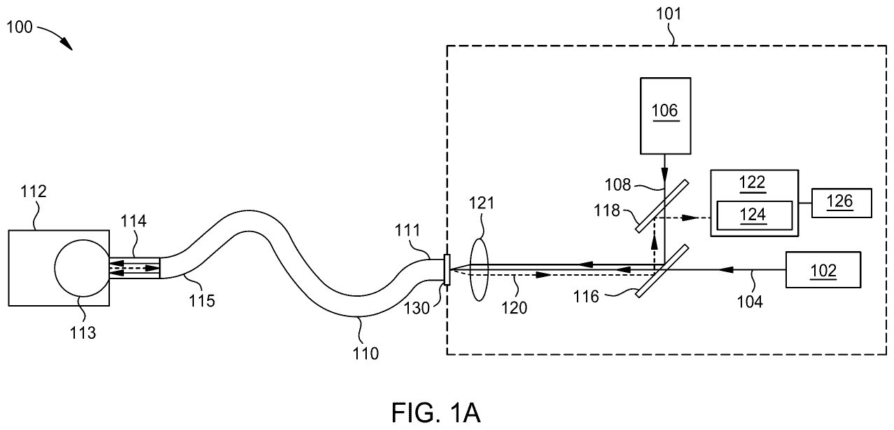

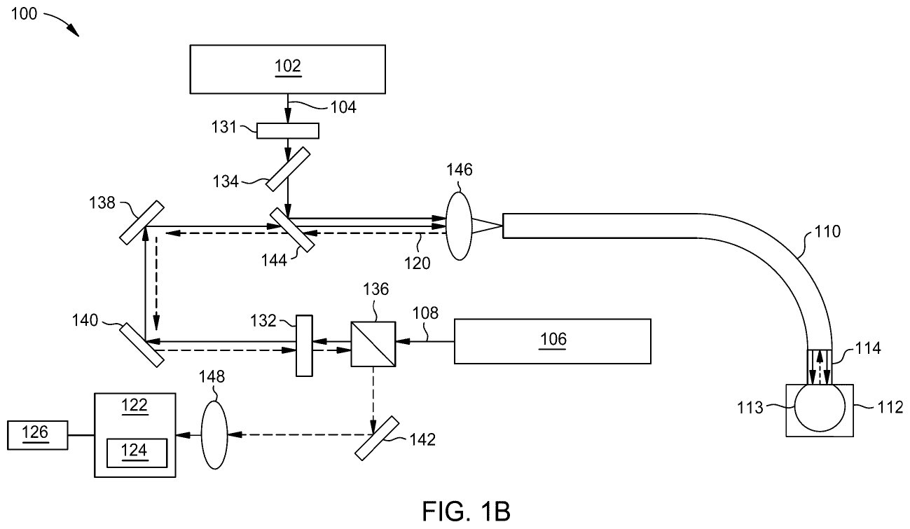

FIG. 1A illustrates a system 100 for performing laser power measurement, in accordance with certain embodiments of the present disclosure. The system 100 includes a surgical laser system 101, which may be operably coupled to and/or in communication with a surgical console, such as a surgical console for ophthalmic surgical procedures. The surgical laser system 101 comprises a first laser source 102 configured to generate a first laser beam 104, and in certain embodiments, a second laser source 106 configured to generate a second laser beam 108. Generally, the system 100 enables measurement of the power of the first laser beam 104 as generated by the first laser source 102 of the surgical laser system 101 in real time.

In some embodiments, the first laser source 102 is a treatment laser source configured to generate the first laser beam 104 for treatment of a patient. The first laser beam 104 may be used for cutting and/or emulsifying material during a surgical operation. For example, the first laser beam 104 may be used as a treatment beam for performing various functions during ophthalmic surgical procedures (e.g., vitreoretinal procedures, glaucoma surgeries, cataract surgeries, etc.).

In some embodiments, the first laser beam 104 generated by the first laser source 102 is an ultraviolet (“UV”) (<350 nm) (nanometers) laser light. In some embodiments, the first laser beam 104 is an infrared (“IR”) (780-4000 nm) laser light, such as a mid-IR laser light. In some embodiments, the first laser beam 104 is an argon blue-green laser light (488 nm), a Nd-YAG (neodymium-doped yttrium aluminum garnet) laser light (532 nm) such as a frequency-doubled Nd-YAG laser light, a krypton red laser light (647 nm), or any other suitable type of laser light for ophthalmic surgery. In some embodiments, the first laser beam 104 has a wavelength of about 500 nm.

In some embodiments, the first laser source 102 may generate and propagate the first laser beam 104 having a pulse rate within a range of about 100 hertz (Hz) and 10 kilohertz (kHz). In some embodiments, the first laser source 102 may generate and propagate the first laser beam 104 having a pulse rate within a range of about 10 kilohertz (kHz) and about 500 kHz, or between about 1 kHz and about 1500 Hz. Other pulse rate ranges are contemplated as well. In some examples, the first laser source 102 produces a nanosecond, a picosecond, or a femtosecond first laser beam 104. In some embodiments, the first laser source 102 is a continuous wave (CW) laser source that can be switched to a pulsed mode during a calibration procedure.

Returning now to FIG. 1A, in certain embodiments, the surgical laser system 101 also includes the second laser source 106 configured to generate the second laser beam 108. The second laser beam 108 may, in certain embodiments, function as a test laser beam for measuring power levels of the system 100. In some embodiments, the second laser beam 108 may be configured to further operate as a source of illumination of a surgical site, for aiming, or the like.

In some embodiments, the second laser beam 108 generated by the second laser source 106 is a visible (380-780 nm) or IR (780-1000 nm) laser light. For example, in certain embodiments, the second laser beam 108 comprises a 640 nm laser light. However, other spectrums/ranges (e.g., 400 nm to 4 μm) are further contemplated for the second laser beam 108.

In some embodiments, the second laser source 106 may generate and transmit the second laser beam 108 having a pulse rate within a range of about 100 hertz (Hz) and 10 kilohertz (kHz). In some embodiments, the second laser source 106 may generate and transmit the second laser beam 108 having a pulse rate within a range of about 10 kilohertz (kHz) and about 5 MHz (megahertz), or between about 1 kHz and about 1500 Hz. Other pulse rate ranges are contemplated as well. In some examples, the second laser source 106 produces a nanosecond, a picosecond, or a femtosecond second laser beam 108. In some embodiments, the second laser source 106 may produce a continuous coherent or semi-continuous second laser beam 108. For example, the second laser source 106 may produce a continuous wave second laser beam 108 at low power.

In some embodiments, the first laser beam 104 and the second laser beam 108 may be generated by a single laser source of the surgical laser system 101 that is configured to produce two or more types of laser beams, or laser beams having different characteristics. For example, in some embodiments, the first laser beam 104 and the second laser beam 108 may be generated by the first laser source 102. In some other embodiments, the first laser beam 104 and the second laser beam 108 may be generated by the second laser source 106.

The system 100 further includes an optical fiber 110 having a proximal end 111 configured to be removably coupled to a port 130 of the surgical laser system 100. The optical fiber 110 may be configured to proximally receive and distally propagate both of the first laser beam 104 and the second laser beam 108 generated by the first laser source 102 and the second laser source 106, respectively, which may be disposed adjacent to the proximal end 111 of the optical fiber 110. The optical fiber 110 may include any suitable type of optical fiber configured to transmit light energy along a length of the optical fiber 110. In some embodiments, the optical fiber 110 may, at least in part, be made of germanium oxide-based glass, sapphire, fluoride, zirconium fluoride, and/or silica. The optical fiber 110 may include a single material, a blend of materials, may have different regions of different materials, etc. However, any suitable materials or space for the efficient propagation of laser beams 104 and 108 are contemplated. The optical fiber 110 may be at least partially clad, single-clad, double clad, multi-clad, or may be unclad. In embodiments with cladding, the cladding may be concentric with one or more cores of the optical fiber 110. In some embodiments, the first laser beam 104 and/or the second laser beam 108 may be propagated through the optical fiber 110 via a cladding.

In some embodiments, the optical fiber 110 has a single core structure. In such embodiments, the first laser beam 104 and the second laser beam 108 may be propagated along the same core of the optical fiber 110. In other embodiments, the optical fiber 110 has a multi-core structure. In such embodiments, the first laser beam 104 and the second laser beam 108 may be propagated along the same core or different cores of the optical fiber 110.

Generally, the optical fiber 110 may be rigid or flexible. In some embodiments, the optical fiber 110 may be straight or tapered. In some embodiments, a diameter of the optical fiber 110 is between about 100 μm (micrometers) and about 400 μm, such as between about 100 μm and about 300 μm, such as about 100 μm and about 200 μm, such as about 200 μm and about 400 μm, such as about 200 μm and about 300 μm, such as about 300 μm and about 400 μm. In some embodiments, the optical fiber 110 may have different regions having similar or different geometries to one another. In such embodiments, the different regions may comprise one or more pieces of optical fiber butt-coupled to each other.

The optical fiber 110 may also be configured to distally receive and proximally propagate a reflected portion 120 of the second laser beam 108 that is reflected back by, and into, a distal end 115 of the optical fiber 110 during performance of power level measurements. The reflected portion 120 may be passed through the optical fiber 110 along the same core or a different core of the optical fiber 110 as at least one of the first laser beam 104 or the second laser beam 108 being propagated through the optical fiber 110 in the opposite direction.

In some embodiments, the optical fiber 110 includes an optical fiber tip 114 disposed at the distal end 115 of the optical fiber 110 opposite the first laser source 102 and/or the second laser source 106. Generally, the first laser beam 104 and the second laser beam 108 may be transmitted (i.e., emitted) distally from the optical fiber tip 114 after being propagated through the optical fiber 110. The optical fiber tip 114 may be fabricated of similar or different construction from another portion of the optical fiber 110. For example, the optical fiber tip 114 may vary from another portion of the optical fiber 110 in material, material properties, optical properties, geometry, or the like. For example, the optical fiber tip 114 may be rigid while another portion of the optical fiber 110 may include a flexible portion to allow for positioning of the optical fiber tip 114 relative to a test material 112 or a surgical site. In some embodiments, the optical fiber tip 114 comprises a lens or window for facilitating transmission of the first laser beam 104 and the second laser beam 108 distally from the optical fiber 110. In some embodiments, the lens or window may comprise sapphire. In some embodiments, the optical fiber tip 114 is configured to be disposed within, or be integrated with, a handpiece of a surgical tool, such as an ophthalmic surgical laser probe. In some embodiments, the material of the optical fiber tip 114 is selected to be strong enough to withstand the shock of repeatedly expanding and collapsing bubbles, and/or to not chemically interact with the test material 112. One example of such a material includes sapphire.

In some embodiments, the optical fiber 110 and/or the system 100 may further include one or more optical elements configured to direct, re-direct, filter, polarize, focus, collimate, split, or otherwise manipulate the first laser beam 104, the second laser beam 108, and/or the reflected portion 120 of the second laser beam 108. For example, in FIG. 1A, a first dichroic mirror 116 and a second dichroic mirror 118 are depicted. Generally, the dichroic mirrors 116 and 118 may facilitate either the reflection or transmission of laser beams depending on their wavelengths. In FIG. 1A, the first dichroic mirror 116 is depicted as facilitating transmission of the first laser beam 104 and re-direction (e.g., reflection) of the second laser beam 108 into the optical fiber 110, while also facilitating re-direction of the proximally-travelling reflected portion 120 of the second laser beam 108 toward the second dichroic mirror 118. The second dichroic mirror 118 then re-directs the reflected portion 120 into an optical detector 122 (or any suitable type of signal detector).

In the illustrated embodiment of FIG. 1A, a focal lens 121 is also shown. The focal lens 121 may be configured to focus at least one of the first laser beam 104 or the second laser beam 108 onto the optical fiber 110 at or through the port 130. For example, the focal lens 121 may be configured to focus at least one of the first laser beam 104 or the second laser beam 108 onto a core of the optical fiber 110.

The system 100 further includes the optical detector 122, which is configured to receive the reflected portion 120 and generate an optical detector output based on the reflected portion 120. In some embodiments, the optical fiber 110 is configured to direct the reflected portion 120 of the second laser beam 108 to the optical detector 122 indirectly (e.g., via an air, or other, gap). In other embodiments, the optical fiber 110 may direct the second laser beam 108 to the optical detector 122 through direct contact transmission. The optical detector 122 may include a sensor 124 (e.g., photodiode or other energy sensitive detector element) capable of detecting the reflected portion 120 of the second laser beam 108 incident at the optical detector 122 and further capable of generating an optical detector output.

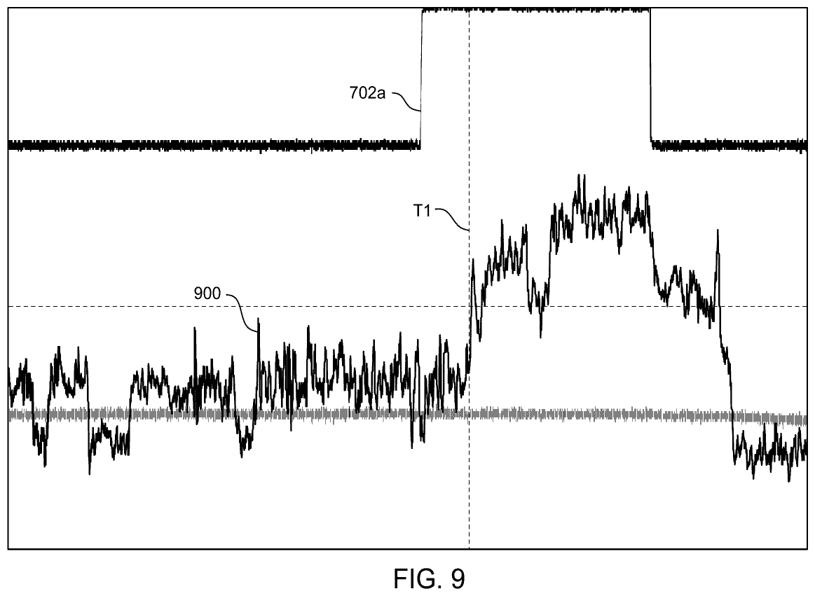

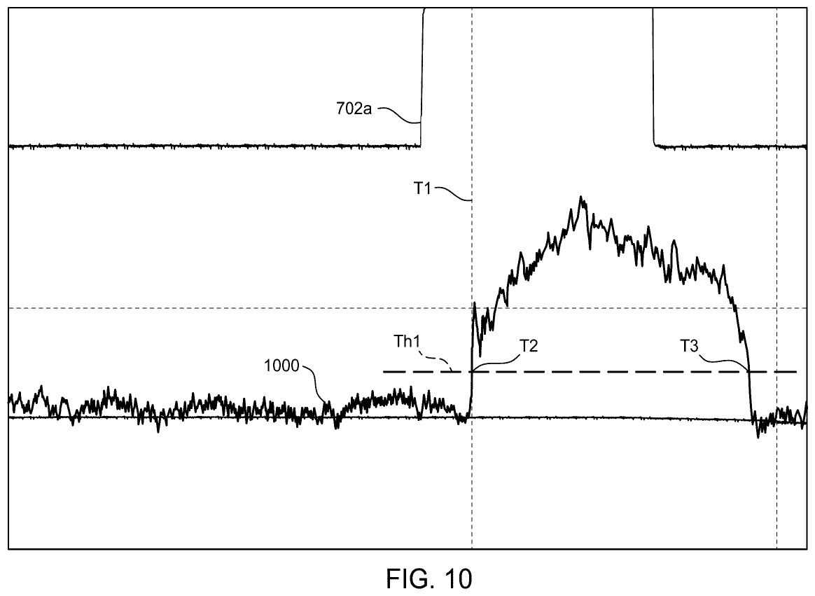

In some embodiments, the optical detector output may be electrically amplified. In other embodiments, the optical detector output may pass through a high-pass filter to separate the transient back-reflection signal from a DC (direct current) baseline, or the high-pass filter may be used prior to or after the amplifier, or between amplifier stages. The optical detector 122 is coupled to a controller 126 that is configured to receive and analyze the optical detector output from the optical detector 122 corresponding to the detected reflected portion 120 and determine various metrics/characteristics of the reflected portion 120. Such metrics/characteristics of the reflected portion 120 are utilized to determine power levels of laser light generated by at least the first laser source 102 at the optical fiber tip 114, as described in further detail below. Please note that although the optical detector 122 and controller 126 are shown as integrated components of the surgical laser system 101 in FIG. 1A, the optical detector 122 and controller 126 may be separate components operably coupled with the surgical laser system 101, such components of a surgical console operably coupled with the surgical laser system 101. A low pass filter may be used to remove unnecessary noise from the back-reflection signal (see FIGS. 9 and 10 for the shape of signals before and after low-pass filtering).

The system 100 may also include, or be used in combination, with a test material 112, into which the optical fiber 110 is configured to direct the first laser beam 104 and the second laser beam 108 for performing power level measurement of the first laser beam 104. In some embodiments, during use, the optical fiber 110 may be positioned to be at least partially disposed within the test material 112. In other embodiments, during use, the optical fiber 110 may be positioned to contact only a surface of the test material 112. The optical fiber 110 is configured to emit the first laser beam 104 and the second laser beam 108 from the optical fiber 110 into the test material 112. In some embodiments, the test material 112 may include a liquid such as water, saline, balanced salt solution (BSS), or the like. In some embodiments, the test material 112 may include a viscoelastic material. Other examples may include liquid, semi-liquid, and/or semi-solid materials that form a transient bubble upon delivery of laser energy to the test material. In some embodiments, the test material 112 is a disposable or single-use material. In some embodiments, the test material 112 may be a reusable or multi-use material.

To measure the power level of laser light generated by the first laser source 102, the optical fiber tip 114 is placed into or adjacent the test material 112, and the first laser source 102 is activated to generate the first laser beam 104. The first laser beam 104 is received at the proximal end of optical fiber 110 and is propagated distally through the optical fiber 110 for transmission from the optical fiber tip 114 into the test material 112. Upon receipt of the first laser beam 104 by the test material 112, the thermal energy of the first laser beam 104 causes the test material 112 to vaporize, or otherwise change in state or form, resulting in a bubble 113 or cavity within the test material 112 and adjacent to the optical fiber tip 114. Continued transmission of the first laser beam 104 into the test material 112 causes the resultant bubble 113 to expand before collapsing. In certain embodiments, each subsequent emission/firing of the first laser beam 104 from the first laser source 102 may generate a corresponding bubble. Generally, differing levels of power of the first laser beam 104 will cause different bubble characteristics or bubble formation profiles for the bubble 113.

Simultaneously with the first laser beam 104, the second laser beam 108 is generated and propagated distally through the optical fiber 110 and out of the optical fiber tip 114. The second laser beam 108 is thus conveyed to an interface between the test material 112 and the optical fiber tip 114 of the optical fiber 110. As the bubble 113 forms and expands due to the thermal energy of first laser beam 104, the second laser beam 108 will be subjected to a change in index of refraction at the interface as the interface shifts from, for example, a solid-liquid (e.g., sapphire-BSS) interface to a solid-vapor (e.g., sapphire-vaporized BSS) interface. For example, the index of refraction before formation of the bubble 113 may be approximately equal to 1.33 between the optical fiber 110 and the test material 112. Once the bubble 113 is formed, the index of refraction may change to approximately 1.0 at the interface of the optical fiber 110 and the vapor within the bubble 113.

The change in the index of refraction at the distal end of the optical fiber 110 results in a change of the Fresnel coefficients of the distal end of the optical fiber 110 and, thus, the optical behavior of the second laser beam 108 at the end of the optical fiber 110, whereby an increased amount of second laser beam 108 is back-reflected through the optical fiber 110. For example, with the bubble 113 present, a larger portion of the second laser beam 108 may be transiently reflected by the optical fiber tip 114 proximally through the optical fiber 110 while a smaller portion of the second laser beam 108 may pass into the test medium. A portion of the transmitted part of the laser beam may be further reflected from the inner surface of the bubble 113 back into the fiber.

The change in optical behavior of the second laser beam 108 is detected by the sensor 124 of optical detector 122, which continuously or non-continuously monitors/detects for the reflected portion 120 of the second laser beam 108 and sends an optical detector output to the controller 126. The controller 126 coupled therewith may then analyze the detected signal to determine metrics/characteristics of the reflected portion 120, which can be correlated with predefined or predetermined bubble characteristics or bubble formation profiles and thus, a power of the first laser beam 104.

FIG. 1B illustrates another embodiment of the system 100 of FIG. 1A. As mentioned above, additional optical components and/or relays are also contemplated for use with the system 100. The illustrated embodiment of FIG. 1B includes additional optical components in the form of wave plates (e.g., half wave plate 131, quarter wave plate 132), polarizing elements (polarizer 134, polarizing cube 136), reflectors (e.g., reflector 138, 140, 142), dichroic elements (e.g., dichroic 144), lenses (e.g., focusing lenses 146, 148, collimators, etc.), and the like. In some examples, the optical components may be used to facilitate power modification, laser light isolation, transmission of identified wavelengths, etc. One or more components may include coatings (e.g., anti-reflective coating), materials, gratings, films, etc. to separate wavelengths, isolate the laser from the back-reflected beams or the like. In some embodiments, physical structures may be used to transmit light. In other embodiments, light may be transmitted through free space. In some embodiments, light may be transmitted via a combination of physical structures and free space.

Note that other surgical laser systems are also contemplated for use with the present systems and methods for real-time laser power measurement. Such surgical laser systems include those described in U.S. patent application Ser. No. 17/662,148 (U.S. Patent Publication No. 20220354692) entitled “Surgical Laser System with Illumination”, filed May 5, 2022, which is herein incorporated by reference in its entirety.

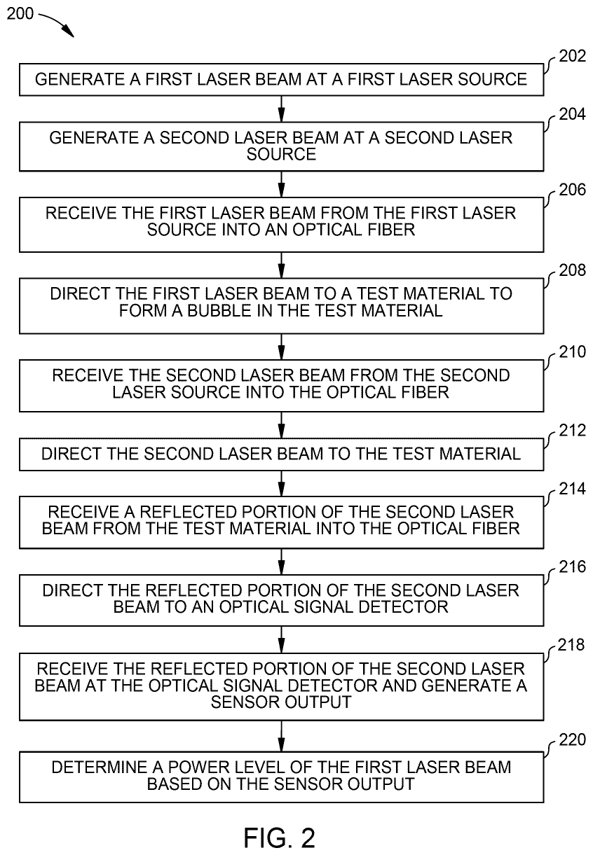

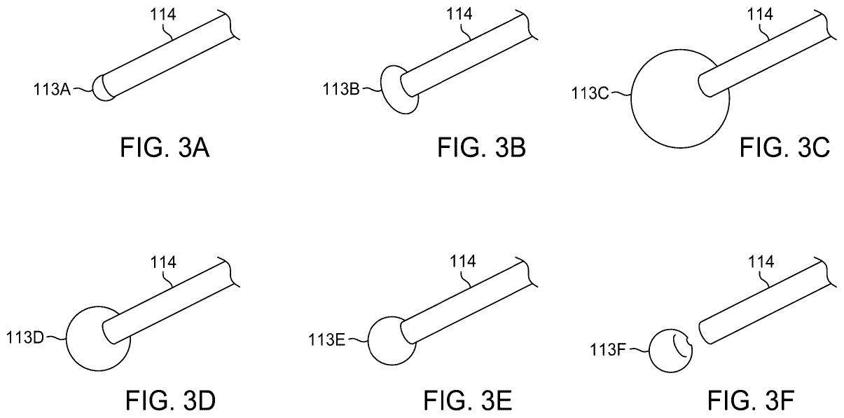

FIG. 2 illustrates a method 200 of performing laser power measurement with the system 100, according to certain embodiments described herein. FIGS. 3A-3F illustrate one or more operations of the method 200. Accordingly, FIG. 2 and FIGS. 3A-3F are herein described together, where appropriate, for clarity.

Turning to FIG. 2, at block 202 of the method 200, the first laser beam 104 is generated by the first laser source 102.

At block 204, the second laser beam 108 is generated by the first laser source 102 or a second laser source 106. In some embodiments, the second laser beam 108 is generated simultaneously with the first laser beam 104. In some embodiments, the second laser beam 108 is generated sequentially with the first laser beam 104. For example, the second laser beam 108 may be generated prior to generating the first laser beam 104, or vice versa.

At block 206 of the method 200, the first laser beam 104 is received from the first laser source 102 into the optical fiber 110, which may be an optical fiber having one or more cores and/or claddings for simultaneously or sequentially propagating the first laser beam 104 and second laser beam 108. In other embodiments, the optical fiber may be an unclad fiber, such as an unclad sapphire rod or fiber.

At block 208, the optical fiber 110 directs (e.g., propagates) the first laser beam 104 to the test material 112 to form a bubble 113 in the test material 112. As described above, the test material 112 may include a liquid such as water, saline, balanced salt solution (BSS), or the like. The formation of the bubble 113, for a short period of time, changes the refractive index of the test material 112 and therefore, the reflection coefficient of the interface between the optical fiber tip 114 of the optical fiber 110 and the test material 112.

FIGS. 3A-3F illustrate the formation of the bubble 113 during block 208. In particular, in FIG. 3A, the first laser beam 104 is transmitted from the optical fiber tip 114 into the test material 112. As the energy from the first laser beam 104 interacts with the test material 112, a first stage bubble 113A begins to form. Formation of the first stage bubble 113A is marked by a separation of the test material 112 from the optical fiber tip 114.

In FIG. 3B, a second stage bubble 113B continues to expand, thereby increasing in volume relative to the first stage bubble 113A. The expansion of the second stage bubble 113B is due to expanding vapor within the bubble 113 as caused by the energy of the first laser beam 104.

In FIG. 3C, a third stage bubble 113C reaches a peak volume as vapor pressure and dynamic movement of the bubble 113 reach an equilibrium with the pressure of the test material 112.

In FIG. 3D, a fourth stage bubble 113D begins to collapse, thus reducing in volume and allowing the test material 112 to draw closer to the optical fiber tip 114 as the energy from the bubble 113 disperses.

In FIG. 3E, a fifth stage bubble 113E continues to reduce in volume and the fifth stage bubble 113E begins to separate from the optical fiber tip 114 during the collapse. In FIG. 3F, a sixth stage bubble 113F is fully separated from the optical fiber tip 114 with test material 112 fully interstitial between the sixth stage bubble 113F and the optical fiber tip 114.

Turning back now to FIG. 2, at block 210 of the method 200, the second laser beam 108 is propagated into the optical fiber 110 by the second laser source 106. At block 212, the second laser beam 108 is directed through the optical fiber 110 and toward the test material 112, as previously described in FIG. 1A. In some embodiments, the second laser beam 108 may be carried in a same core of the optical fiber 110 as the first laser beam 104, or in a different core in examples where the optical fiber 110 comprises a multi-core arrangement. In some embodiments, the optical fiber 110 may be clad, and the first laser beam 104 and/or second laser beam 108 are propagated in a cladding.

At block 214 of the method 200, a portion (e.g., reflected portion 120) of the second laser beam 108 is back-reflected from the interface of the optical fiber tip 114 and the test material 112 and is received by the optical fiber 110. As the bubble 113 is formed, the bubble 113 changes the reflection coefficient of the interface between the optical fiber tip 114 and the test material 112, thereby creating a transient modulation of the back-reflected portion 120. In certain embodiments, some of the second laser beam 108 may be reflected from within the bubble 113 to return into the optical fiber 110.

At block 216, the reflected portion 120 of the second laser beam 108 is propagated back through the optical fiber 110 and is directed to the optical detector 122 (such as by using one or more optics or relays).

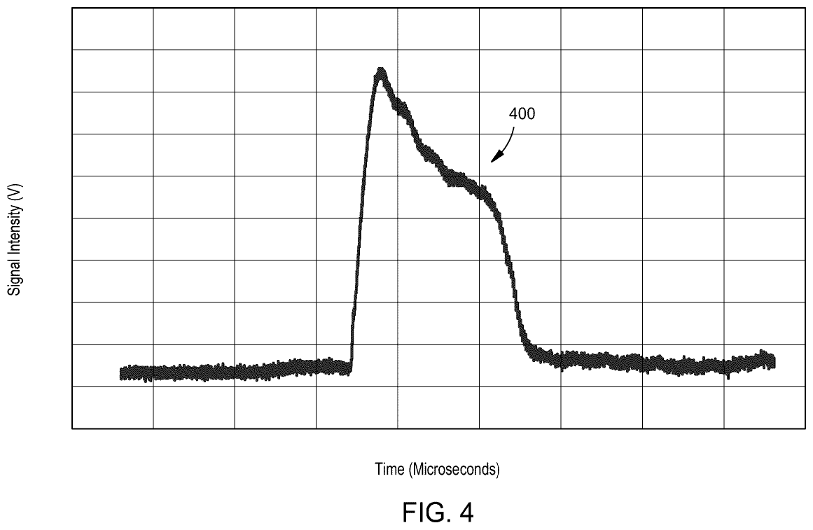

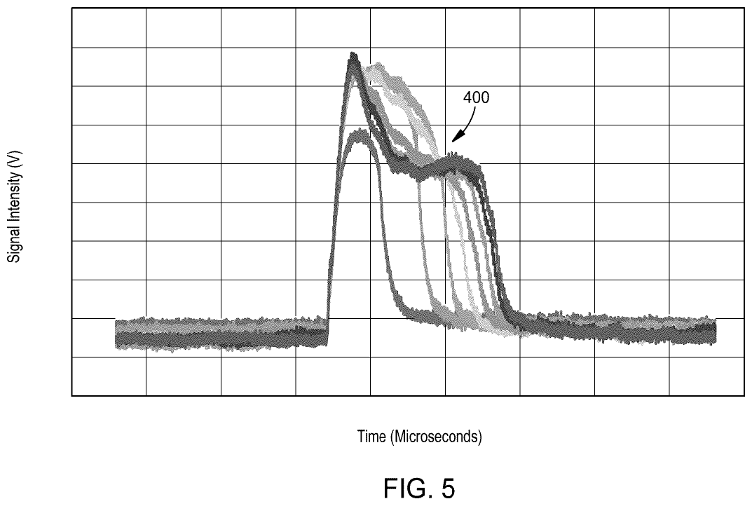

At block 218, the method 200 includes receiving the reflected portion 120 of the second laser beam 108 at the sensor 124 and generating, by the sensor 124 and/or optical detector 122, an optical detector output (e.g., a signal) based on the received reflected portion 120. In some embodiments, the optical detector output is a signal profile (e.g., a measured reflection time signal as described below in FIGS. 4 and 5).

In some embodiments, the optical detector output may be electrically amplified. In other embodiments, the electrical detector output may pass through a high-pass filter to separate the transient back-reflection signal from the DC baseline, or the high-pass filter may be used in front or after the amplifier, or between amplifier stages.

At block 220, the method 200 includes determining a power level of the first laser beam 104, in real time, based on the optical detector output. For example, in some embodiments, various features and/or characteristics of the signal profile of the reflected portion 120 may be correlated to features and/or characteristics of predetermined or defined signal profiles corresponding to one or more power levels of the first laser beam 104. Such features and/or characteristics may include a number of peaks, a duration of peaks, a rise time, a fall time, etc. In some embodiments, a signal profile of the reflected portion 120 corresponds to the lifetime of a single bubble 113 formed as a result of firing the first laser beam 104 into the test material 112. Accordingly, in some embodiments, the power level of the first laser beam 104 may be determined based on a time duration component of the reflected portion 120 as determined from the optical detector output.

Turning now to FIGS. 4 and 5, exemplary signal profiles for the reflected portion 120 of the second laser beam 108 during the formation and collapse of the bubble 113 in the test material 112, as detected by the optical detector 122, are illustrated according to certain embodiments described herein. While particular shapes of the signal profiles are shown, other shapes may also result and also provide power measurement capability.

In the example of FIG. 5, a single signal profile 400 is shown, corresponding to the formation and collapse of a single bubble 113 as caused by the first laser beam 104 at a certain power level. The signal profile 400 comprises a plot of signal voltage values over time for the reflected portion 120 of the second laser beam 108. In the illustrated example, the signal profile 400 for the reflected portion 120 includes a first peak 402 (and decline) corresponding to the first stage bubble 113A of FIG. 3A. The first peak 402 may be indicative of a slightly higher reflectivity as an interface between the first stage bubble 113A and the test material 112 may still be proximate to the optical fiber tip 114, but is beginning to separate therefrom, thus causing the subsequent drop in reflection following the first peak 402.

After the first peak 402, the signal profile 400 steadily rises, which corresponds to the formation of the second stage bubble 113B as vapor within the bubble 113 expands and a greater portion of the second laser beam 108 is reflected by the optical fiber tip 114 at formation of the second stage bubble 113B. In the illustrated example, the signal profile 400 includes a second peak 404 corresponding to the formation of the third stage bubble 113C. The third stage bubble 113C may represent a maximum volume of the bubble 113 at which vapor pressure within the bubble 113 is at equilibrium with the resistance of the test material 112. Because the vapor within the bubble 113 may be at the lowest density during the third stage bubble 113C, the signal profile 400 may be highest at this second peak 404 based on the index of refraction at the optical fiber tip 114. As the bubble 113 progresses to the fourth stage bubble 113D, the signal profile 400 may begin to decrease with a greater drop off occurring as the bubble 113 collapses more rapidly through the fifth stage bubble 113E. In the illustrate embodiment, the separation of the bubble 113 relative to the optical fiber tip 114 in the sixth stage bubble 113F may return a steady-state value at the optical detector 122 indicative of an end of lifecycle of the bubble 113.

In the example of FIG. 5, a graph of eight different signal profiles 400 are shown, wherein each of the eight illustrated signal profiles 400 comprises a plot of signal voltage values over time. Each different signal profile 400 may correspond to a different power level output by the first laser source 102 when generating the first laser beam 104. In the illustrated example, it can be seen that an increase in output power (mW) (milliwatt) for the first laser source 102 resulted in a longer time trace for the corresponding signal profile 400. As further shown, increasing the output power also yielded signal profiles 400 with higher intensity peaks (e.g., corresponding to higher levels of collection of the light back reflected from the curved surface of the bubble) of the second laser beam 108.

Once desired characteristics/features of a signal profile 400 are determined, such as a pulse width or duration of the signal profile 400, such characteristics/features may be mapped to one or more predetermined correlation curves to determine the power level of the first laser beam 104. In some embodiments, the correlation curve may be at least partially dependent on a phase transition enthalpy of the test material 112, a diameter of the optical fiber 110, a pulse wavelength and/or duration of the first laser source 102, etc.

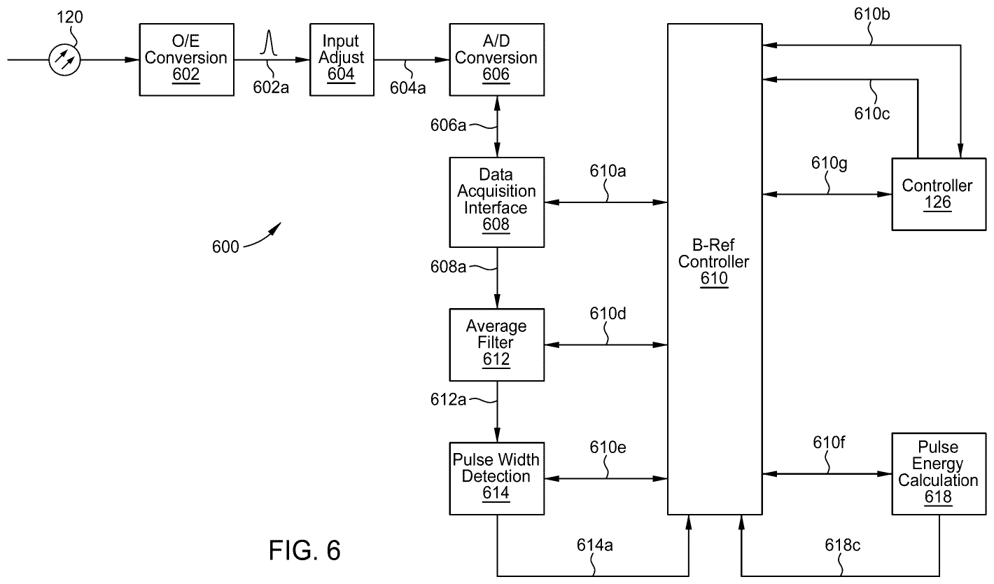

Referring to FIG. 6, illustrated system 600 may be implemented to process measurements of back reflected light. The system 600 is further configured to identify pulses in measurements of back reflected light and estimate energy delivered to patient tissue based on widths of the identified pulses. The system 600 may therefore be used to implement block 220 of the method 200.

For example, the reflected portion 120 of light from the second laser source 106 may be processed by performing an optical to electrical (O/E) conversion operation 602, which may be carried out at the sensor 124. The output of the O/E conversion operation 602 may be an analog electrical signal 602a. The analog electrical signal 602a may be processed by the system 600 at the sensor 124 by performing an input adjustment operation 604. The input adjustment operation 604 may involve one or more analog operations on the analog electrical signal 602a, such as low pass filtering to prevent aliasing during a subsequent analog-to-digital (A/D) conversion operation 606. The input adjustment operation 604 may include other operations such as scaling, biasing, or other pre-processing to compensate for properties of the sensor 124.

The output of the input adjustment operation 604 is an adjusted signal 604a that is input to the A/D conversion operation 606 of the system 600. The A/D conversion operation 606 includes sampling the adjusted signal 604a at a sampling frequency to obtain a time-series of samples 606a, each corresponding to a different timestep. The A/D conversion operation 606 may be performed by an A/D converter. Low pass filtering of the input adjustment operation 604 may have a cutoff frequency less than half of the sampling frequency. For example, an external user interface or other input scheme may send data acquisition commands to the controller 126. The controller 126 may initiate the A/D conversion operation 606 by sending a control signal or command 610g to the B-ref controller 610. The B-ref controller 610 may then send commands to operation 606 through the data acquisition interface 608.

The samples 606a may be input to a data acquisition interface 608 of the controller by the B-ref controller 610. The system 600 is primarily configured to measure pulse width of pulses in the adjusted B-ref signal, the pulses being the result of pulses emitted by the first laser source 102. The data acquisition interface 608 may therefore selectively store a portion of the samples 606a output by the A/D conversion operation 606 that are likely to contain a representation of a pulse. For example, the data acquisition interface 608 may read and store samples 606a only when commanded to do so by a B-ref controller 610. For example, the B-ref controller 610 may use a data acquisition control signal 610a to control the start and end of reading and storing of samples 606a by the data acquisition interface 608.

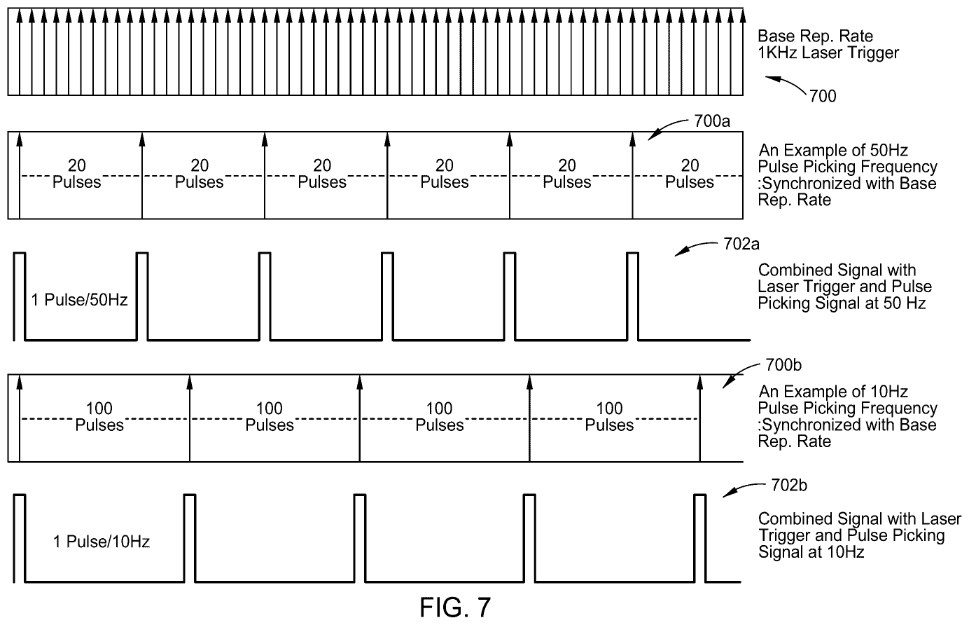

For example, referring to FIG. 7 while still referring to FIG. 6, the first laser source 102 may have a base repetition rate at which the first laser source 102 is capable of or in fact does emit pulses 700. The base repetition rate may be controlled by a trigger frequency of a laser trigger signal 610b that is used to control the first laser source 102. The laser trigger signal 610b may be generated by the B-Ref controller 610 or provided to the B-Ref controller 610 to facilitate synchronization with the first laser source 102. In the illustrated example, the base repetition rate is 1 kHz. In most applications, the base repetition rate is too rapid and therefore only some pulses 700a are selected for transmission out of the first laser source 102 and into the optical fiber 110. For example, a pulse picking signal 610c may control which pulses are output by the first laser source 102. The pulse picking signal 610c may be generated by the B-Ref controller 610 or provided to the B-Ref controller 610 to facilitate synchronization. The frequency of pulses in the pulse picking signal 610c will control which pulses 700a are actually output by the first laser source and into the optical fiber 110. In some embodiments, the controller 126 receives the laser trigger signal 610b and generates the pulse picking signal 610c based on laser trigger signal 610b.

An example combination of the laser trigger signal 610b and the pulse picking signal is illustrated as combined signal 702a, and in such an example, pulses generated by the first laser source 102 during pulses in the combined signal 702a will be output into the optical fiber 110. For example, for a 1 kHz base repetition rate, every 20^{th} pulse 700a may be selected by the combined signal 702a having a pulse picking frequency of 50 Hz that is synchronized with the base repetition rate. In another example, every 100^{th} pulse 700b may be selected by the combined signal 702a having a pulse picking frequency of 10 Hz to achieve the illustrated combined signal 702b.

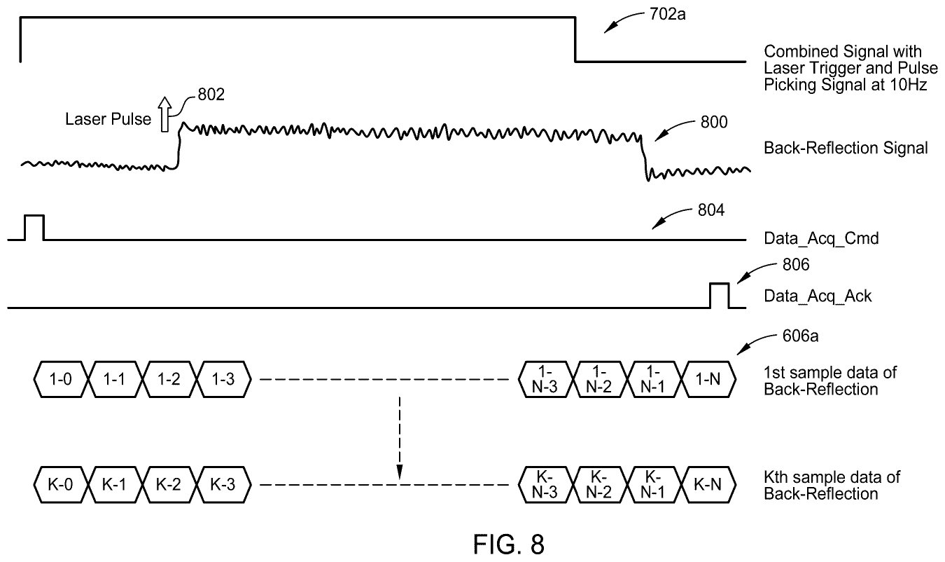

Referring to FIG. 8, for a given pulse in the combined signal 702a (or the signal 702b in a like manner), the first laser source 104 will emit a laser pulse 802 into the optical fiber 110. Upon incidence on patient tissue, the laser pulse 802 may cause one or more bubbles to form (see FIGS. 3A to 3F and corresponding description, above), which may cause scattering of light emitted by the second laser source 106. The back-reflection signal 800 may therefore exhibit a pulse subsequent to incidence of the laser pulse 802 on the patient tissue. The illustrated back-reflection signal 800 may correspond to the analog electrical signal 602a or the adjusted signal 604a.

The B-ref controller 610 may control the data acquisition interface 608 in synchronization with generation of the laser pulse 802. In certain embodiments, at or around (e.g., within one sample period) the start of the pulse in the combined signal 702a, the B-ref controller 610 may output a command 804 to start data acquisition. For example, the rising edge of a pulse in the combined signal 702a may invoke generation of the command 804 to start data acquisition. In certain embodiments, at or around the end of the pulse in the combined signal 702a, the B-ref controller 610 may output a command 806 to end data acquisition. For example, as shown in FIG. 8, the command 806 may be output subsequent to a falling edge of a pulse in the combined signal 702a. In certain embodiments, the system 600 may refrain from processing samples output by the A/D conversion operation 606 after the command 806 and before the next command 804. Refraining from processing these samples reduces processing requirements of the system 600.

The delay between the command 804 to start data acquisition and the command 806 to end data acquisition may be a tuned parameter selected by a designer based on an estimated lifetime of bubbles resulting from emitting laser pulses. Accordingly, the command 806 may be invoked prior to the falling edge of a pulse in the combined signal 702a where the duration of pulses in the combined signal 702a is longer than the estimated lifetime of bubbles resulting from emitting laser pulses. In some instances, there is a predictable delay between the rising edge of a pulse in the combined signal 702a and the commencement of back reflection resulting from bubbles. Accordingly, the command 804 may be output by B-ref controller 610 subsequent to the rising edge of a pulse in the combined signal 702a.

The estimated lifetime of bubbles resulting from emitting a laser pulse from the first laser source 102 may be determined experimentally. For example, the delay between the commands 804, 806 may be equal to an adjustment factor plus the average measured lifetime of bubbles resulting from emitting laser pulses from one or more first laser sources 102 into tissue of one or more patients.

Samples 606a output by the A/D conversion stage 606 may then be stored by the data acquisition interface 608 and/or passed on by the data acquisition interface 608 for processing by a subsequent operation. Following the next rising edge of a pulse in the combined signal 702a, another N samples may be stored and/or passed on in a like manner, such as the illustrated K-0 to K-N samples. N may be an integer representing the index of a stored sample and K may be an integer representing the set of samples read for a given pulse in the combined signal 702a. The number N is dependent on the expected lifetime of bubbles and the sampling period of the A/D conversion stage 606.

Referring again to FIG. 6, a portion of the samples 606a that are stored and/or passed on by the data acquisition interface 608 (samples 608a) may be received by an average filter stage 612. The average filter stage 612 receives the samples 608a and outputs a smoothed set of samples 612a in which each sample in samples 612a is an average of a set of contiguous samples in the samples 608a. For example, let S[n] represent each sample 608a and let A[n] represent each sample 612a. A[n] may be calculated as a function of samples S[n−a] to S[n+b], where a and b are integers defining the number of samples averaged (a+b+1) and the lookback (a) at which samples are selected from S[n]. For example, A[n] may be calculated as an average of samples S[n−a] to S[n+b].

The operation of the average filter stage 612 may be controlled by the B-Ref controller 610. For example, the B-Ref controller 610 may output an average control signal 610d to the average filter stage 612. For example, the B-ref controller 610 may select the number of samples averaged (e.g., values for a and b), a weighting function (e.g., triangular moving average, exponential moving average, etc.), or other parameters defining averaging of samples.

The samples 612a may be input to a pulse width detection operation 614. The pulse width detection operation 614 determines the first and last samples of a contiguous group of samples 612a that are above a threshold. The pulse width detection operation 614 then derives the pulse width 614a from the indexes of the first and last samples in the contiguous group.

The pulse width detection operation 614 may be controlled by the B-Ref controller 610. For example, the B-Ref controller 610 may output a pulse width control signal 610e to the pulse width detection operation 614. For example, the B-ref controller 610 may select the threshold used to detect samples corresponding to pulses resulting from bubbles.

FIGS. 9 and 10 illustrate operation of the average filter operation 612 and the pulse width detection operation 614. Referring specifically to FIG. 9, plot 900 is a plot of raw samples, such as samples 606a or 608a of FIG. 6. FIG. 9 further includes a plot of the combined signal 702a and a labeled time T1 at which the back reflected light rises upon formation of one or more bubbles in response to a pulse 802 from the first laser source 102. As is apparent in FIG. 9, the plot 900 shows a large amount of noise that makes it difficult to identify the time T1.

FIG. 10 illustrates a plot 1000 of samples 612a. As is readily apparent, noise prior to time T1 is substantially reduced, enabling identification of the start and end of a pulse corresponding to formation of one or bubbles in response to a pulse 802. For example, a threshold Th1 may be defined. A pulse width may also be defined as the difference between a time T2 at which the B-ref Average Data rises above the threshold Th1 and a time T3 at which the B-Ref Average Data falls below the threshold Th1. T2 may be defined as a first index of a first sample above threshold Th1 and T3 may be defined as an index of the first sample below the threshold Th1 following the first sample. The pulse width detection operation 614 may calculate the pulse width in seconds as (T3−T1)*dT, where dT is the sample period of the A/D conversion operation 606.

As is apparent from a comparison of the plot 900 to the plot 1000, the threshold Th1 may be lower than some of the peaks smoothed by the average filter operation 612. The threshold Th1 may also be selected to be higher than noise present in the output of the average filter operation 612 that is not the result of bubbles caused by a laser pulse 802.

Returning to FIG. 6, a pulse width 614a, as determined by the pulse width detection operation 614, may be output to the B-Ref controller 610. The pulse width 614a may be output as calculated or may be further processed. For example, a plurality of pulse widths may be calculated, and a statistical characterization thereof may be output (average, minimum, maximum, standard deviation, etc.) as the pulse width 614a.

The pulse width 614a may be processed by a pulse energy calculation operation 618. For example, the B-Ref controller 610 may input the pulse width 614a to the pulse energy calculation operation 618. The pulse energy calculation operation 618 may be controlled by the B-ref controller 610 using a pulse energy control signal 610f.

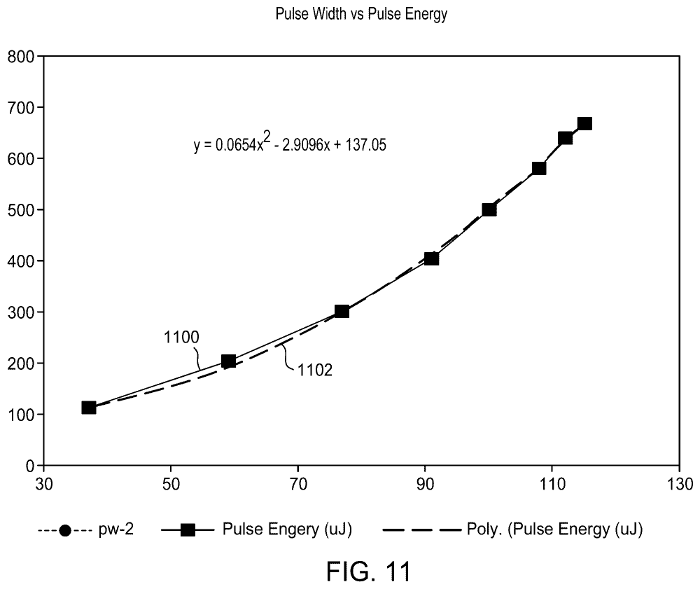

For example, referring to FIG. 11, the pulse energy calculation operation 618 estimates energy in a laser pulse 802 as a function of the pulse width 614a. The pulse width may be measured as described above for pulses with known pulse energy. A curve fit may then be generated by a designer or user that calculates the pulse energy as a function of pulse width.

FIG. 11 illustrates plots of pulse energy (vertical axis) with respect to pulse width (horizontal axis). Plot 1100 is a plot of actual pulse energy with respect to measured pulse width. Plot 1102 is a plot of estimated pulse energy based on measured pulse width. For example, the plot 1102 may be a result of performing curve fitting with respect to measured pulse widths to attempt to match the plot 1100. In the illustrated example, polynomial curve fitting is used and the function obtained by curve fitting is y=0.0654x²-2.9096x+137.05, where x is the measured pulse width and y is the estimated pulse energy. In other embodiments, a lookup table relating pulse width to measured pulse energy may be used by the pulse energy calculation operation 618 with interpolation being performed to estimate pulse energy for pulse widths for which a pulse energy measurement is not recorded in the lookup table. Any other approach for relating an input variable to an output variable may be used to estimate the pulse energy for a given pulse width.

As is readily apparent, close correspondence between the estimated and actual pulse energies may be obtained. Accordingly, the pulse width may be used by a human or software component to estimate pulse energy delivered to patient tissue for at least most applications. In particular, the pulse width provides more than enough accuracy to detect connectivity loss between the first laser source 102 and the optical fiber tip 114.

Referring again to FIG. 6, an estimated pulse energy 618c as determined by the pulse energy calculation operation 618 may be output by the pulse energy calculation operation 618 to the B-Ref controller 610. The estimated pulse energy 618c may be used in various ways. In some embodiments, a representation of the estimated pulse energy 618c is displayed on a display device to enable a surgeon to verify proper function of the system 100. In some embodiments, the B-Ref controller 610 exchanges control signals 610g with the controller 126. For example, the B-Ref controller 610 may cause the controller 126 to increase or decrease pulse energy of the first laser source 102 based on the estimated pulse energy 618c.

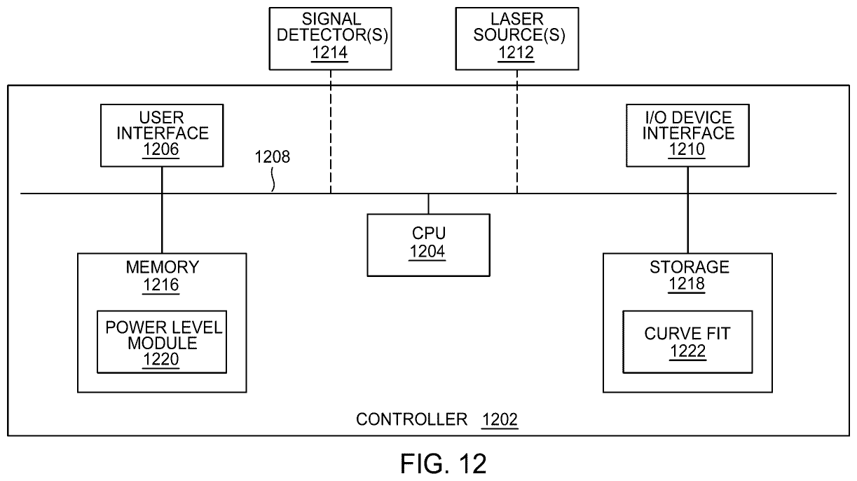

FIG. 12 illustrates a schematic diagram of a controller 1202, according to embodiments disclosed herein. The controller 1202 is generally representative of the controller 126 and/or system 600 described above and, in some embodiments, may be integrated with or operably coupled with a surgical console. In some embodiments, the controller 1202 includes, without limitation, a user interface 1206 and at least one I/O (Input/Output) device interface 1210, which may allow for the connection of various I/O devices (e.g., keyboards, displays, mouse devices, pen input, etc.) to the controller 1202. The controller 1202 may be in wired or wireless communication with one or more laser sources 1212 (e.g., the first laser source 102 and/or the second laser source 106 of FIG. 1A) and one or more optical detectors 1214 (e.g., optical detector 122 of FIG. 1A having sensor 124) via the interconnect 1208. In some embodiments, in addition to or separately from the controller 126, the one or more laser sources 1212 and/or the one or more optical detectors 1214 may be integrated with or operably coupled with a surgical console.

The controller 1202 further includes a CPU 1204 (Central Processing Unit), a memory 1216, and a storage 1218. The CPU 1204 is configured to retrieve and execute programming instructions stored in the memory 1216. Similarly, the CPU 1204 may retrieve and store application data residing in the memory 1216. The interconnect 1208 transmits programming instructions and application data, among the CPU 1204, I/O device interface 1210, user interface 1206, memory 1216, storage 1218, laser source(s) 1212, optical detector(s) 1214, etc. The CPU 1204 may include a single CPU, multiple CPUs, a single CPU having multiple processing cores, and the like. The memory 1216 may be random access memory, and the storage 1218 may be a disk drive. Moreover, the memory 1216 and/or storage 1218 may be any type of a readily available memory, such as random access memory (RAM), read only memory (ROM), floppy disk, hard disk, solid state, flash memory, magnetic memory, or any other form of digital storage, local or remote. In certain embodiments, the memory 1216 and/or storage 1218 include instructions, which when executed by the CPU 1204, can affect determinations/measurements of a power levels of the laser source(s) 1212 based on data received from the optical detectors 1214. In certain embodiments, the CPU 1204, memory 1216, and storage 1218 may be the main processor and memory of controller 1202.

In the embodiment of FIG. 12, the CPU 1204 of the controller 1202 may include an integrated circuit capable of performing logic functions. In this manner, the CPU 1204 is in the form of a standard integrated circuit package with power, input, and output pins. In other embodiments, the CPU 1204 is a microprocessor. In other cases, the CPU 1204 is not a programmable microprocessor, but instead is a special purpose controller.

In the embodiment of FIG. 12, the controller 1202 receives signals from one or more optical detectors 1214. These signals, for example, may include optical detector output corresponding to reflected light received at sensor(s) of the one or more optical detectors 1214.

As shown, the storage 1218 includes a curve fit 1222, such as a curve fit generated as described above with respect to FIG. 11 that relates or maps measured pulse width to estimated pulse energy.

As shown, the memory 1216 includes a power level module 1220. The power level module 1220 may implement the functionality of the B-ref controller 610 in order to measure pulse width of back reflected signals and obtain an estimated pulse energy therefrom as described above. In certain embodiments, after a power of the laser source(s) 1212 is determined by the power level module 1220, the controller 1202 may output the determined power to a user graphical display or other I/O device in communication with the controller 1202 via the I/O device interface 1210.

In summary, embodiments of the present disclosure include systems and methods for laser power measurement, and more particularly, systems and methods for laser power measurement in a sterile operating environment. In certain embodiments described herein, power measurement is performed utilizing two laser sources: 1) a first laser source configured to generate a first laser beam (treatment laser); and 2) a second laser source configured to generate a second laser beam (test laser). The first laser beam generates a bubble in a test material (e.g., water, saline, balanced salt solution, gel, etc.) and the second laser beam is reflected back with the reflected portion of the second laser beam being measured to determine a lifetime of the bubble and correlate the lifetime measurement with a power value based on a correlation curve. Laser power measurement using the back reflection of the second laser beam allows for testing in a sterile environment and embodiments may allow for testing during a surgical operation. Testing of the laser power level can identify equipment issues including improper assembly, faulty components, misalignment due to movement or use prior to or during a surgical operation, and the like. Thus, the methods and systems described herein enable not only improved safety of testing in a sterile environment, but also improve the ability to test power levels of a laser prior to and during an operation without introducing contaminants.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module. The following claims are not intended to be limited to the embodiments shown herein but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system for laser power and pulse energy measurement, the system comprising:
a first laser source configured to generate a first laser beam in a pulsed form;
a second laser source configured to generate a second laser beam;
an optical fiber configured to:
receive the first laser beam from the first laser source;
output the first laser beam from a distal end of the optical fiber;
receive the second laser beam from the second laser source;
output the second laser beam from a distal end of the optical fiber;
receive a reflected portion of the second laser beam that is reflected back into the distal end of the optical fiber; and
direct the reflected portion of the second laser beam to an optical detector;
the optical detector, configured to:
receive the reflected portion of the second laser beam from the optical fiber; and
generate an optical detector output based on the reflected portion of the second laser beam; and
a controller, configured to:
determine one or more widths of one or more pulses in the optical detector output; and
determine an estimated energy for one or more pulses in the first laser beam based on the one or more widths.

2. The system of claim 1, wherein the first laser beam comprises an infrared laser light and the second laser beam comprises visible laser light.

3. The system of claim 1, wherein the controller is further configured to:
process samples of the optical detector output in synchronization with a signal invoking transmission of the one or more pulses in the first laser beam to determine the one or more widths of one or more pulses in the optical detector output.

4. The system of claim 1, wherein the controller is further configured to:
process first samples of the optical detector output for first time periods including each pulse of the one or more pulses in the first laser beam to identify the one or more pulses in the optical detector output; and
refrain from processing second samples of the optical detector output for second time periods not including the one or more pulses in the first laser beam.

5. The system of claim 1, wherein the controller is further configured to:
smooth the optical detector output to obtain a smoothed output; and
process the smoothed output to determine the one or more widths of the one or more pulses in the optical detector output.

6. The system of claim 5, wherein the controller is further configured to smooth the optical detector output by performing a moving average of the optical detector output.

7. The system of claim 5, wherein the controller is further configured to process the smoothed output to determine the one or more widths of the one or more pulses in the optical detector output by identifying one or more groups of contiguous samples in the smoothed output that are above a threshold.

8. The system of claim 1, wherein the controller is further configured to generate the estimated energy for the one or more pulses in the first laser beam based on the one or more widths by evaluating a polynomial function with respect to the one or more widths.

9. A method comprising:
generating, by a first laser source, a first laser beam in a pulsed form;
generating, by a second laser source, a second laser beam;
receiving, by an optical fiber, the first laser beam from the first laser source;
directing, by the optical fiber, the first laser beam to patient tissue to form a bubble in the patient tissue;
receiving, at the optical fiber, the second laser beam from the second laser source;
directing, by the optical fiber, the second laser beam to the patient tissue;
receiving, at the optical fiber, a reflected portion of the second laser beam from an interface of the patient tissue and the optical fiber;
directing, by the optical fiber, the reflected portion of the second laser beam to an optical detector;
receiving, at the optical detector, the reflected portion of the second laser beam from the optical fiber;
determining, by a controller in communication with the optical detector, one or more widths of one or more pulses in an output of the output of the optical detector; and
determining, by the controller, an estimated energy for one or more pulses in the first laser beam based on the one or more widths.

10. The method of claim 9, wherein the first laser beam comprises an infrared laser light and the second laser beam comprises visible laser light.

11. The method of claim 9, further comprising:
processing, by the controller, samples of the output of the optical detector in synchronization with a signal invoking transmission of the one or more pulses in the first laser beam to determine the one or more widths of one or more pulses in the output of the optical detector.

12. The method of claim 9, further comprising:
processing, by the controller, first samples of the output of the optical detector for first time periods including each pulse of the one or more pulses in the first laser beam to identify the one or more pulses in the output of the optical detector; and
refraining, by the controller, from processing second samples of the output of the optical detector for second time periods not including the one or more pulses in the first laser beam.

13. The method of claim 9, further comprising:
smoothing, by the controller, the output of the optical detector to obtain a smoothed output; and
processing, by the controller, the smoothed output to determine the one or more widths of the one or more pulses in the output of the optical detector.

14. The method of claim 13, further comprising processing, by the controller, the smoothed output to determine the one or more widths of the one or more pulses in the output of the optical detector by identifying one or more groups of contiguous samples in the smoothed output that are above a threshold.

15. The method of claim 9, further comprising generating, by the controller, the estimated energy for the one or more pulses in the first laser beam based on the one or more widths by evaluating a polynomial function with respect to the one or more widths.
